(19) United States
(12) Patent Application Publication
(10) Pub. No.: US 2025/0329173 A1
(43) Pub. Date: Oct. 23, 2025

(54) MULTIMODAL SENSOR AND VISION-BASED DRIVER ALCOHOL IMPAIRMENT DETECTION SYSTEM

(71) Applicant: VINAI ARTIFICIAL INTELLIGENCE APPLICATION AND RESEARCH JOINT STOCK COMPANY, Ha Noi (VN)

(72) Inventors: Duy Duc Nguyen, Ha Noi (VN); Thanh Lam Nguyen, Ha Noi (VN); The De Vu, Ha Noi (VN); Viet Tien Tran, Ha Noi (VN); Duc Toan Bui, Ha Noi (VN); Thanh Vuong Cap, Ha Noi (VN); Hai Hung Bui, Ha Noi (VN)

(21) Appl. No.: 18/642,354

(22) Filed: Apr. 22, 2024

(51) Int. Cl. G06V 20/59; A61B 5/16; A61B 5/18; G01N 33/497; G06V 10/80

(52) U.S. Cl. CPC G06V 20/597; G01N 33/4972; G06V 10/803; A61B 5/163; A61B 5/18

(57) ABSTRACT

A driver impairment detection system includes an on-board camera mounted to a vehicle. An on-board sensor is configured to detect a presence of alcohol in an ambient environment of the operator. An on-board computing system is mounted to the vehicle. The on-board computing system includes a processor or controller module that is configured to: communicate with an artificial intelligence engine, receive image data from the camera, receive environmental alcohol vapor data from the on-board sensor, and provide the image data and the environmental alcohol vapor data to the artificial intelligence engine. The artificial intelligence is configured to: detect whether eyes of an operator of the vehicle indicate impairment while driving, and determine whether a presence of alcohol in the ambient environment is emanating from a passenger in the vehicle other than the operator.

### FIELD OF THE INVENTION

The present invention relates in general to detection systems. More particularly, the invention is directed to a multimodal sensor and vision-based driver alcohol impairment detection system.

### BACKGROUND OF THE INVENTION

In the field of vehicle operation monitoring, systems exist to detect impaired operators. Some systems use active detection devices, (for example, breathalyzers) which attempt to thwart would-be inebriated drivers by requiring the driver to blow into a sensor. Failed tests deactivate the vehicle ignition system for some time. Other systems monitor operator behavior and try to detect whether the operator is exhibiting signs of impairment. There are some systems that detect whether alcohol is present in the vehicle. However, these systems lack any precision and can result in false flags. The false indications can be impractical to those that are merely in the company of others that have drunk alcohol when the systems disable use of the vehicle or worse yet, identify the driver as operating the vehicle while impaired even if the river has not drunk. The aforementioned scenario is particularly troublesome for those vehicle operators that provide ride services for others that have drunk alcohol. Those systems that merely identify the presence of alcohol in the air inadvertently stymie unimpaired drivers providing a service to the community.

### SUMMARY OF THE INVENTION

In a first aspect, a driver impairment detection system is disclosed. The system includes an on-board camera mounted to a vehicle. An on-board sensor is configured to detect a presence of alcohol in an ambient environment of the operator. An on-board computing system is mounted to the vehicle. The on-board computing system includes a processor or controller module that is configured to: communicate with an artificial intelligence engine, receive image data from the camera, receive environmental alcohol vapor data from the on-board sensor, and provide the image data and the environmental alcohol vapor data to the artificial intelligence engine. The artificial intelligence is configured to: detect whether eyes of an operator of the vehicle indicate impairment while driving, and determine whether a presence of alcohol in the ambient environment is emanating from a passenger in the vehicle other than the operator.

In a second aspect, a method of detecting driver impairment in a vehicle is disclosed. The method includes receiving, by a processor, image data from an on-board camera of the vehicle. The processor receives environmental alcohol vapor data from an on-board sensor of the vehicle. The image data and the environmental alcohol vapor data are provided to an artificial intelligence engine. The artificial intelligence engine determines whether eyes of an operator of the vehicle indicate impairment while driving. The artificial intelligence engine determines whether a presence of alcohol in the ambient environment is emanating from a passenger in the vehicle other than the operator.

In a third aspect, a computer program product for detecting driver impairment in a vehicle is disclosed. The computer program product comprises a non-transitory computer readable storage medium having computer readable program code embodied therewith. The computer readable program code is configured, when executed by a processor, to: receive, by the processor, image data from an on-board camera of the vehicle. The processor receives environmental alcohol vapor data from an on-board sensor of the vehicle. The image data and the environmental alcohol vapor data are provided to an artificial intelligence engine. The artificial intelligence engine determines whether eyes of an operator of the vehicle indicate impairment while driving. The artificial intelligence engine determines whether a presence of alcohol in the ambient environment is emanating from a passenger in the vehicle other than the operator.

These and other features and advantages of the invention will become more apparent with a description of preferred embodiments in reference to the associated drawings.

### DESCRIPTION OF THE DRAWINGS

FIG. 1 is a diagrammatic top view of a multimodal sensor and vision-based driver alcohol impairment detection system in a vehicle cabin according to an embodiment of the subject technology.

FIG. 2 is a block diagram of a system for detecting impairment of an operator in a vehicle in accordance with an embodiment of the subject technology.

FIG. 3 is a block diagram of an artificial intelligence engine in accordance with an embodiment of the subject technology.

FIG. 4 is a block diagram of a computing device in accordance with an embodiment of the subject technology.

FIG. 5 is a flowchart of a method for detecting impairment of an operator of a vehicle in accordance with an embodiment of the subject technology.

FIG. 6 is a flowchart of a method of generating warning alerts for impairment in accordance with an embodiment of the subject technology.

### DETAILED DESCRIPTION OF THE PREFERRED EMBODIMENTS

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. Like or similar components are labeled with identical element numbers for case of understanding.

In general, and referring to the Figures, embodiments provide a system and method for detecting impairment of an operator of a vehicle. An embodiment of the subject technology uses a multimodal configuration to determine when an operator of a vehicle is impaired. In one embodiment, image data (the basis for a one mode of detection) and alcohol vapor data in the vehicle (the basis for another mode of detection) are analyzed by a computing device to determine when the operator is impaired (for example, drunk). Embodiments may use artificial intelligence to analyze the data to determine when the operator is impaired, and to identify when the source of potential impairment is from another occupant of the vehicle that is not the operator.

### Example Vehicle Cabin Environment

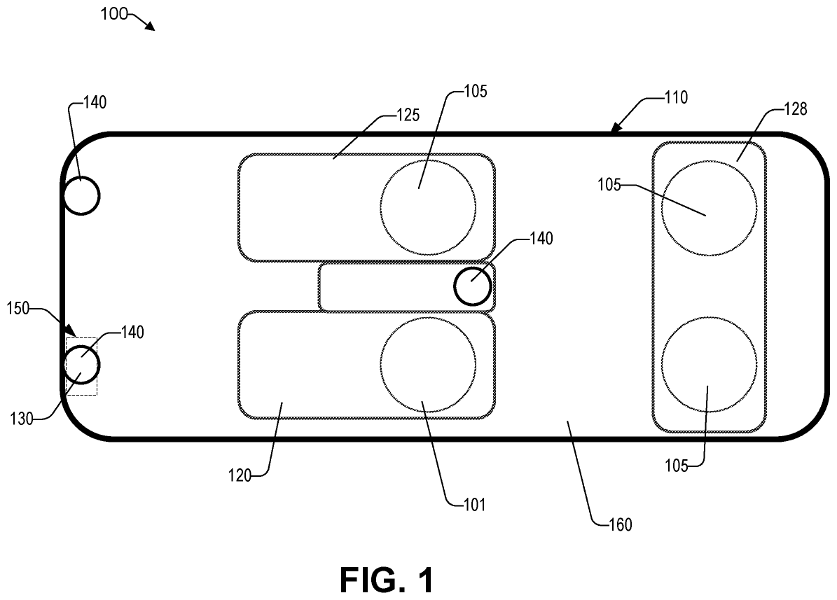

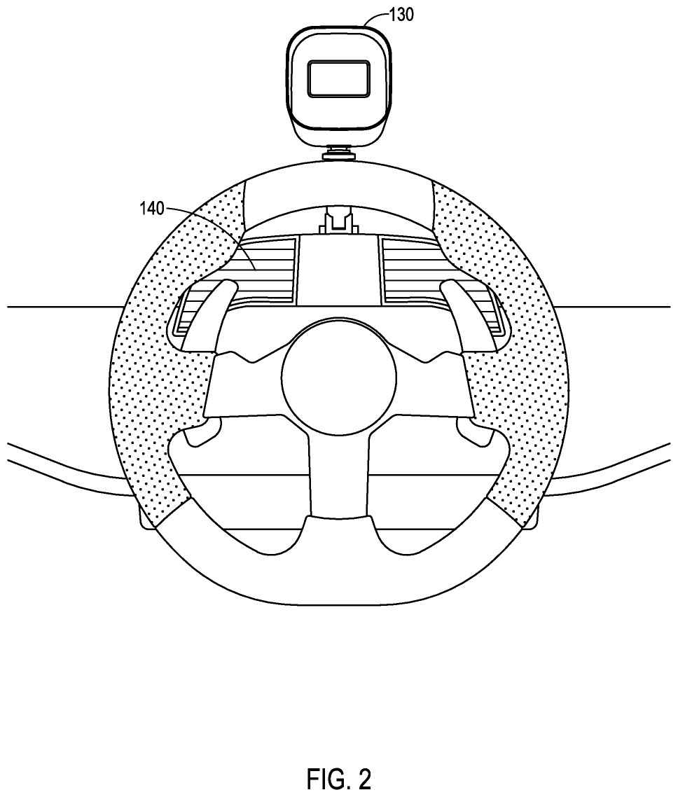

FIG. 1 shows a system 100 for determining a source of impairment in a vehicle 110 according to an embodiment. For sake of illustration the vehicle 110 represents an automobile, however it will be understood that the vehicle 110 may be other vehicle types. The vehicle 110 includes a cabin 160 which may be enclosed. For points of reference, the cabin 160 may include a driver's seat 120, a front side passenger seat 125, and one or more rear passenger seats 128. Element 101 in the driver's seat 120 represents the position of an operator of vehicle 110. Elements 105 represent positions for passengers in their seats 125 or 128. The system 100 includes a computing system 150 (represented by the box in dashed line). The computing system 150 may be a pre-existing on-board computing system or may be a dedicated computing system that is separate from a vehicle's original equipment manufacturer computing system. The computing system 150 may be hidden from view (for example, inside a dashboard as represented by the dashed line). Some embodiments may have an impairment detection module that includes the computing system 150 combined with a camera 130 and an ambient environment alcohol vapor sensor 140 (not visible in this view). The impairment detection module may be a multimedia head unit (MHU) (See for example, FIG. 2). As may be appreciated, detection of impairment may be performed locally in the vehicle without the need for remote computing. In some embodiments, multiple alcohol sensors 140 may be positioned throughout the cabin apart from the MHU, and may be connected to the MHU via cabling (for example, USB connections). In the embodiment shown, alcohol sensors 140 may also be positioned in front of the front side passenger seat 125 (for example, on the dashboard) and approximately entered in the cabin 160 (for example, in between the driver seat 120 and front side passenger seat 125 and in proximity of the rear passenger seats 128 (for example, on a rear end of a console)).

As shown in FIG. 2, the MHU may be mounted to a steering wheel or nearby so that the camera 130 captures the face of the driver and the alcohol sensor 140 is within range to detect alcohol vapor from the driver. The camera 130 may be a near infra-red (NIR) device. The camera 130 is positioned in front of the driver to track his/her facial information. The camera 130 interfaces with the MHU using the GMSL protocol. The alcohol sensor 140 may connect to the MHU via USB cable. In some embodiments, the alcohol sensor 140 contains two fans that draw the driver's exhaled breath into an environmental alcohol sensor inside. Data captured by the camera 130 and all the alcohol sensors 140 are provided to the onboard computing system 150 (FIG. 1) with for example, 30 fps of image capture for analysis to determine operator impairment, the source of alcohol vapor, and a warning strategy.

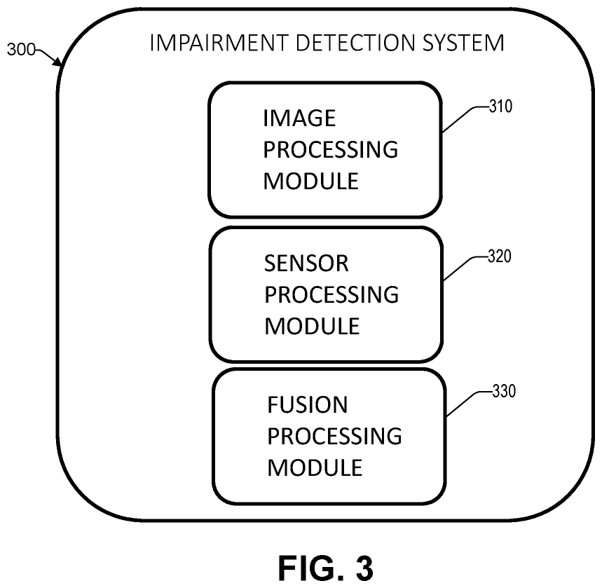

FIG. 3 shows an impairment detection system 300 according to an embodiment. In some embodiments, the impairment detection system 300 resides in the on-board computing system 150. The impairment detection system 300 is an artificial intelligence engine that may include an image processing module 310, a sensor processing module 320, and fusion processing module 330, which in some embodiments may be software modules. The image processing module 310 may be configured to analyze and process the data captured by the camera 130. The sensor processing module 320 may be configured to analyze and process the data captured by the alcohol sensor 140. The fusion processing module 330 combines the output of the two previous modules 310 and 320 to return the final output of the system 300. All the data analysis and determination of operator impairment and the source of alcohol vapors may be performed within the onboard computing system 150.

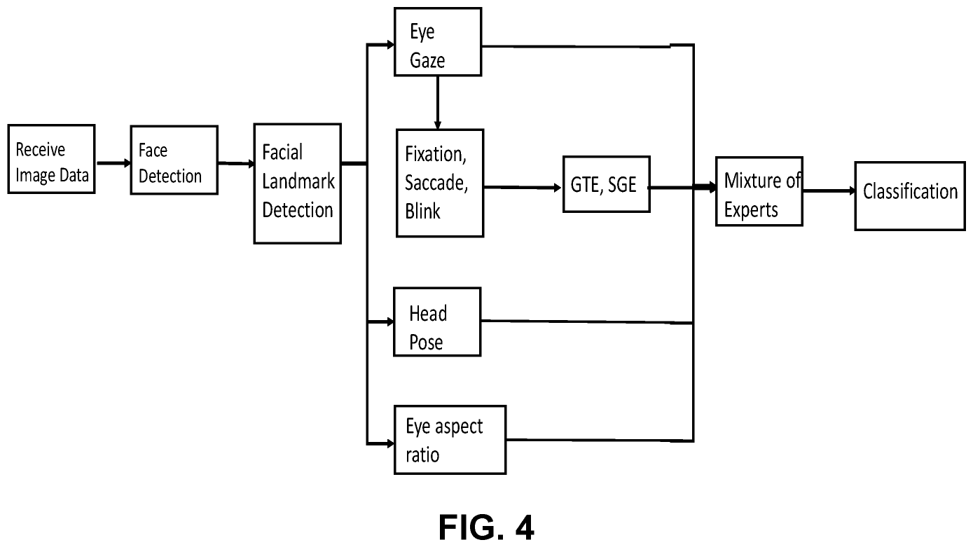

FIG. 4 shows a method of processing image data through the image processing module 310. The method starts with the reception of image data received from the camera 130. The image data is first processed to detect the presence of a face within the image by a face detection module. The detected face is then passed through a landmark detection module to identify for example, sixty-eight facial landmarks. With facial information along with these facial landmarks, the following characteristics may be calculated: aspect ratio of the eyes (EAR), eye movements, head movements, and gaze events. For the EAR, an adaptive self-developed algorithm may be used that can adjust the EAR calculation process depending on each user. The input to this algorithm is the extracted landmarks of the eye region. Head movement may be estimated by calculating the velocity and acceleration of the head across three orientation axes: pitch, yaw, and roll. Eye movement may be estimated by calculating the velocity and acceleration of the eye gaze across two orientation axes: pitch and yaw. For the gaze event, the duration, amplitude, peak velocities, and mean velocities of the fixations and saccades may be calculated. A 1-minute sliding window with 50% overlap may be used to calculate the statistical features such as mean, standard deviation, skewness, kurtosis, power, and 0.05 & 0.95 quantiles from the head movements, eye movements, and gaze event. With the calculated gaze events, gaze transition entropy (GTE) and stationary gaze entropy (SGE) may be calculated which represents the uncertainty of gaze movements. Using the aforementioned processed data, the image processing module 310 may determine whether an attentiveness and an alertness of the operator exceeds a threshold level of impairment. In some embodiments, the impairment detection system 300 is further configured to continuously collect and analyze image data of various operators (for example, different drivers of the same vehicle) from the on-board camera 130, and adapt detection of impairment based on the image data of the various operators.

Given the inherently variable nature of individual responses to alcohol impairment, the instant approach harnesses a machine learning architecture enhanced by a Mixture of Experts (MoE). This advanced architecture is adept at discerning and tailoring the analysis to the unique behavioral patterns of each person. It judiciously determines which specific facets of the data are most indicative of impairment on a case-by-case basis. The foundational input for this deep learning framework is a comprehensive feature vector which is the concatenation of those extracted features above. The output of image processing module after this comprehensive analysis conducted by the machine learning model is a classification scores for three states of impairment from the driver: No-impair, mild-impair and severe-impair.

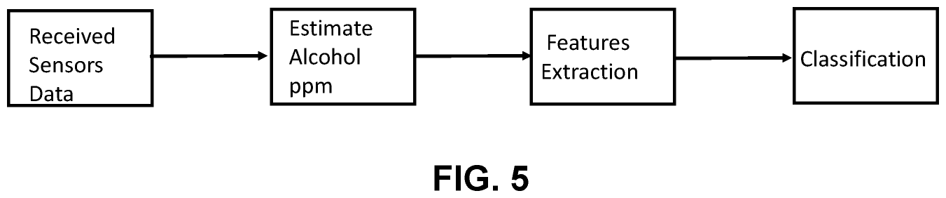

Referring now to FIG. 5, a method of processing alcohol levels in an environment through the sensor processing module 320 is shown according to an embodiment. The method begins by gathering environmental data specifically pertaining to the presence of alcohol, as detected by the alcohol sensors 140. The alcohol sensors 140 may have been finely tuned for optimal performance, with meticulous calibrations applied to their sensitivity parameters and baseline offsets to ensure accuracy. This precise calibration is critical as each alcohol sensor 140 generates an electrical current that is directly influenced by the alcohol detected in the environment. The alcohol sensor 140 is tasked with the intricate process of translating this current into a measurable value, expressed in terms of parts per million (ppm). This quantification process is a pivotal step, converting electrical signals into actionable data that reflects the concentration of alcohol in the cabin. For each sequence of parts per million (ppm) data gathered from the alcohol sensors 140, a two-minute sliding window technique with a 25% overlap may be applied to extract both statistical and temporal characteristics. The statistical features (i.e. “feature extraction”) are obtained through various calculations on the data, such as mean, standard deviation, skewness, kurtosis, minimum, maximum, median, and quartile values. Temporal features, on the other hand, are based on the timing of the data, capturing trends and patterns over time to understand the periodic behavior of the alcohol levels. This extracted data may then be processed using a Recurrent Neural Network (RNN) model to identify the source of alcohol vapor, determining whether the alcohol is emanating from the driver or a passenger. Furthermore, if the driver is identified as the source, the model can differentiate between moderate and severe intoxication levels. In summary, the analysis outcome from the sensor data processing module classifies the driver's condition into sober, moderate, or severe intoxication states.

TABLE 1
Warning signal of our impairment detecting system

vision

| sensor | No-impair | mild-impair | severe-impair |
|---|---|---|---|
| sober | None | None | None |
| moderate | None | Medium | High |
| severe | None | High | High |

The outputs from both the image processing module 310 and the sensor processing module 320 are subsequently combined to generate the final warning signal, an example of which is presented in Table 1. This integrated warning signal from encompasses three distinct levels of alerts: No Alert, Medium Alert, and High Alert. The fusion of data from these two modules 310 and 320 ensures a comprehensive assessment of potential risks, enabling the system to accurately differentiate between situations that require varying degrees of attention.

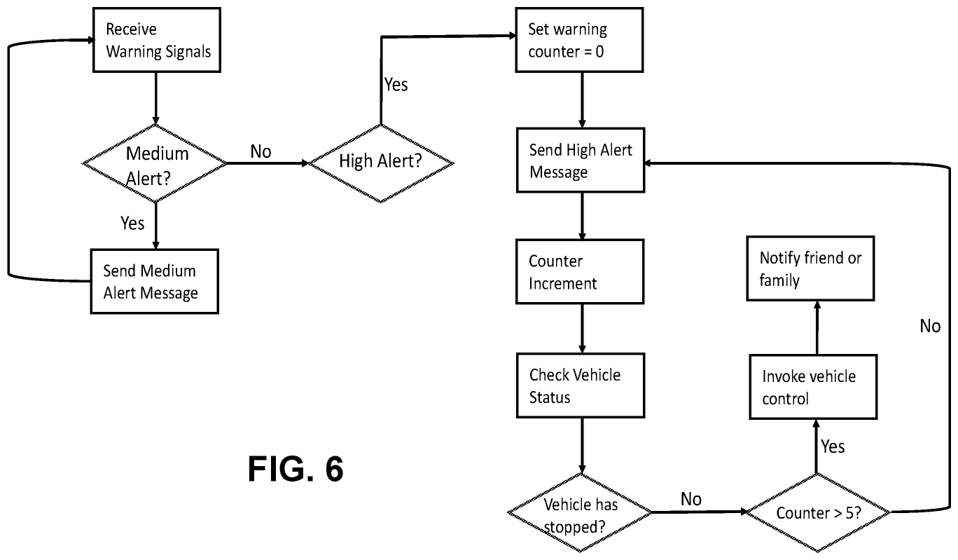

FIG. 6 shows a method of generating warning alerts for impairment according to an embodiment. The warning signals may be returned by the fusion processing module 330. These signals may then be transferred to other parts of the vehicle that are responsible for notifying the operator. Upon receiving warning signals, if the system issues a medium alert, a corresponding alert message (or sound) is transmitted to the driver via a speaker, cautioning that continuing to drive could be hazardous. This alert may be persistently conveyed as long as the medium alert remains active, thereby encouraging the driver to take necessary actions, such as stopping the vehicle. In the event of a high alert, the system may activate a counter to track the number of alerts communicated to the driver. This count increases with each high alert issued. If the driver fails to stop the vehicle following these alerts, the system evaluates the total number of high alerts issued. Should this count surpass a predetermined threshold, set at five for instance, the system then takes control of the vehicle automatically, rendering the vehicle inoperative (or temporarily stopped). In some embodiments, the system may notify a third party, for example, a close relative or friend of the driver (via stored profile data) about the driver's situation. Parameters such as the alert count threshold, alert frequency, sound type, and message content can be adjustable according to the driver's preferences.

Aspects of the disclosed invention are described above with reference to block diagrams of methods, apparatus (systems) and computer program products according to embodiments of the invention. It will be understood that each block of the block diagrams, and combinations of blocks in the flowchart illustrations and/or block diagrams, can be implemented by computer program instructions. These computer program instructions may be provided to the processor of the on-board computing device 150, the impairment detection module 155, a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks in the figures.

Those of skill in the art would appreciate that various components and blocks may be arranged differently (e.g., arranged in a different order, or partitioned in a different way) all without departing from the scope of the subject technology. The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects.

Thus, the claims are not intended to be limited to the aspects shown herein, but are to be accorded the full scope consistent with the language of claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

The word “exemplary” is used herein to mean “serving as an example or illustration.” Any aspect or design described herein as “exemplary” is not necessarily to be construed as preferred or advantageous over other aspects or designs.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used in the description or the claims, such term is intended to be inclusive in a manner similar to the term “comprise” as “comprise” is interpreted when employed as a transitional word in a claim.

1. A driver impairment detection system, comprising:
an on-board camera mounted to a vehicle;
an on-board sensor configured to detect a presence of alcohol in an ambient environment of the operator; and
an on-board computing system mounted to the vehicle, wherein the on-board computing system includes a processor or controller module that is configured to:
communicate with an artificial intelligence engine,
receive image data from the camera,
receive environmental alcohol vapor data from the on-board sensor, and
provide the image data and the environmental alcohol vapor data to the artificial intelligence engine, wherein the artificial intelligence is configured to:
detect whether eyes of an operator of the vehicle indicate impairment while driving, and
determine whether a presence of alcohol in the ambient environment is emanating from a passenger in the vehicle other than the operator.

2. The system of claim 1, wherein the artificial intelligence engine is located locally in the on-board computing system.

3. The system of claim 1, wherein the artificial intelligence engine includes:
an image processing module configured to analyze images of the operator provided by the camera;
a sensor processing module configured to analyze levels of alcohol present detected by the sensor; and
a fusion processing module that combines data analyzed by the image processing module with data analyzed by the sensor processing module and determines a level of impairment of the operator from the combined data analyzed.

4. The system of claim 1, wherein the artificial intelligence engine is configured to track eye movements, an eye state, and a head position of the operator from the image data, and determine whether an attentiveness and an alertness of the operator exceeds a threshold level of impairment.

5. The system of claim 4, wherein the artificial intelligence engine is further configured to analyze the eye movements, the eye state, and the head position of the operator and infer a driving condition of the operator.

6. The system of claim 1, wherein the machine learning module is further configured to: determine an amount of alcohol vapor in part per million (ppm) in an area of the vehicle; and
determine a source of the alcohol vapor based on the determined amount of alcohol vapor in the area of the vehicle.

7. The system of claim 1, wherein the machine learning module is further configured to continuously collect and analyze image data of various operators from the on-board camera, and adapt detection of impairment based on the image data of the various operators.

8. A method of detecting driver impairment in a vehicle, comprising:
receiving, by a processor, image data from an on-board camera of the vehicle;
receiving, by the processor, environmental alcohol vapor data from an on-board sensor of the vehicle;
providing the image data and the environmental alcohol vapor data to an artificial intelligence engine;
detecting, by the artificial intelligence engine, whether eyes of an operator of the vehicle indicate impairment while driving, and
determining, by the artificial intelligence engine, whether a presence of alcohol in the ambient environment is emanating from a passenger in the vehicle other than the operator.

9. The method of claim 8, further comprising analyzing, by the artificial intelligence engine, selected statistical features from the image data.

10. The method of claim 8, further comprising, tracking, by the artificial intelligence engine, eye movements, an eye state, and a head position of the operator from the image data, and determining whether an attentiveness and an alertness of the operator exceeds a threshold level of impairment.

11. The method of claim 10, inferring, by the artificial intelligence engine, a driving condition of the operator, based on the eye movements, the eye state, and the head position of the operator.

12. The method of claim 8, further comprising:
determining an amount of alcohol vapor in part per million (ppm) in an area of the vehicle; and
determining a source of the alcohol vapor based on the determined amount of alcohol vapor in the area of the vehicle.

13. The method of claim 8, further comprising continuously collecting and analyzing, by the artificial intelligence engine, image data of various operators from the on-board camera, and adapting detection of impairment based on the image data of the various operators.

14. A computer program product for detecting driver impairment in a vehicle, the computer program product comprising a non-transitory computer readable storage medium having computer readable program code embodied therewith, the computer readable program code being configured, when executed by a processor, to:
receive, by the processor, image data from an on-board camera of the vehicle;
receive, by the processor, environmental alcohol vapor data from an on-board sensor of the vehicle;
provide the image data and the environmental alcohol vapor data to an artificial intelligence engine;
detect, by the artificial intelligence engine, whether eyes of an operator of the vehicle indicate impairment while driving, and
determine, by the artificial intelligence engine, whether a presence of alcohol in the ambient environment is emanating from a passenger in the vehicle other than the operator.

15. The computer program product of claim 14, further comprising computer readable code configured to analyze, by a machine learning module in the artificial intelligence engine, selected statistical features from the image data.

16. The computer program product of claim 14, further comprising computer readable code configured to track, by the artificial intelligence engine, eye movements, an eye state, and a head position of the operator from the image data, and determine an attentiveness and an alertness of the operator based on the image data.

17. The computer program product of claim 16, further comprising computer readable code configured to infer, by the artificial intelligence engine, a driving condition of the operator, based on the eye movements, the eye state, and the head position of the operator.

18. The computer program product of claim 14, further comprising computer readable code configured to:
determine an amount of alcohol vapor in part per million (ppm) in an area of the vehicle; and
determine a source of the alcohol vapor based on the determined amount of alcohol vapor in the area of the vehicle.

19. The computer program product of claim 14, further comprising computer readable code configured to continuously collect and analyze, by the artificial intelligence engine, image data of various operators from the on-board camera, and adapting detection of impairment based on the image data of the various operators.

20. The computer program product of claim 14, further comprising computer readable code configured to:
determine a first level of impairment of the operator;
trigger a first type of warning correlated to the first level of impairment;
determine whether the first level of impairment of the operator exceeds a threshold value indicating a second, higher level of impairment; and
render the vehicle inoperative when the operator is determined to be in the second, higher level of impairment.
